(19) United States
(12) Patent Application Publication
(10) Pub. No.: US 2019/0297975 A1
(43) Pub. Date: Oct. 3, 2019

(54) ACTIVE VENTING GARMENT USING PIEZOELECTRIC ELEMENTS

(71) Applicant: Intel Corporation, Santa Clara, CA (US)

(72) Inventors: Aleksandar ALEKSOV, Chandler, AZ (US); Sasha N. OSTER, Chandler, AZ (US); Feras EID, Chandler, AZ (US); Shawna M. LIFF, Scottsdale, AZ (US); Thomas L. SOUNART, Chandler, AZ (US); Johanna M. SWAN, Scottsdale, AZ (US); Baris BICEN, Chandler, AZ (US); Valluri R. RAO, Saratoga, CA (US)

(21) Appl. No.: 16/303,386

(22) PCT Filed: Jul. 2, 2016
(86) PCT No.: PCT/US2016/040902

§ 371 Date: Nov. 20, 2018

(51) Int. Cl. A41D 27/28; A41D 13/00; A41D 27/24; A61M 35/00; A61M 5/145

(52) U.S. Cl. CPC A41D 27/285; A41D 13/0015; A41D 27/24; A61M 35/00; A61M 5/145; A61M 2205/3334; A41D 2400/32; A41D 2600/10; A61M 2230/201; A61M 2205/0294; A41D 2300/50

(57) ABSTRACT

Embodiments of the invention include an active venting system. According to an embodiment of the invention, the active venting system may include a substrate having one or more seams formed through the substrate. In order to open the vents defined by the seams through the substrate, a piezoelectric layer may be formed proximate to one or more of the seams. Additional embodiments may include a first electrode and a second electrode that contact the piezoelectric layer in order to provide a voltage differential across the piezoelectric layer. In an embodiment the active venting system may be integrated into a garment. In such an embodiment, the garment may also include an electronics module for controlling the actuators. Additionally, conductive traces may be printed on the garment or sewn into the garment to provide electrical connections from the electronics module to each of the piezoelectric actuators.

### FIELD OF THE INVENTION

Embodiments of the present invention relate generally to the manufacture of devices that include active venting features. In particular, embodiments of the present invention relate to the use of piezoelectric actuators that allow for vents to be controlled in garments and other substrates and methods for manufacturing such devices.

### BACKGROUND OF THE INVENTION

During athletic activity the body increases temperature. In order to maximize performance during such activities, athletes have desired clothing that aids in cooling the body. For example, garments have been designed that include venting features to improve airflow to the skin. However, the venting features are either permanently open (e.g., mesh fabrics) or the venting features require bulky actuators for opening or closing the vents. For example, one or more actuators may be sewn into the textile. The actuator can then displace a portion of the textile by controlling a string or fiber that is attached between the textile and the actuator. Particularly, the actuator winds up the string or fiber to open the vent, and releases the string or fiber to close the vent. In addition to the bulk of the actuator, the actuation of the vent is dependent on the string that may fail. For example, the string may be broken or detach from the textile or actuator during use and/or during washing. In such instances, the vent may no longer be functional.

### BRIEF DESCRIPTION OF THE DRAWINGS

FIG. 1A is a cross-sectional illustration of an active venting system that includes a vent opening that is controlled by a piezoelectric actuator, according to an embodiment of the invention.

FIG. 1B is a cross-sectional illustration of the active venting system in FIG. 1A when the vent is in an opened position, according to an embodiment of the invention.

FIG. 1C is a cross-sectional illustration of an active venting substrate that includes an elastomeric material formed between the substrate and the piezoelectric actuator, according to an embodiment of the invention.

FIG. 2A is a plan view illustration of an active venting system that includes a piezoelectric actuator with electrodes that are formed in a single layer, according to an embodiment of the invention.

FIG. 2B is a cross-sectional illustration of the active venting system in FIG. 2A along the length of one electrode, according to an embodiment of the invention.

FIG. 2C is a cross-sectional illustration of an active venting system along the length of one electrode where the piezoelectric material is formed above the electrode, according to an embodiment of the invention.

FIG. 2D is a cross-sectional illustration of an active venting system along one electrode that includes an elastomeric material formed between the substrate and the piezoelectric actuator, according to an embodiment of the invention.

FIG. 3A is a schematic plan view of a portion of an active venting system that includes seams in a closed position, according to an embodiment of the invention.

FIG. 3B is a schematic plan view of a portion of the active venting system in FIG. 3A in an open position, according to an embodiment of the invention.

FIG. 4A is schematic plan view of a portion of an active venting system that includes seams in a closed position, according to an additional embodiment of the invention.

FIG. 4B is a schematic plan view of a portion of the active venting system in FIG. 4A in an open position, according to an embodiment of the invention.

FIG. 5 is a schematic plan view of a shirt that includes a plurality of active vents that are controlled by an electronics module integrated into the shirt, according to an embodiment of the invention.

FIG. 6A is a cross-sectional illustration of a liquid delivery system that includes an actuatable orifice in a closed position, according to an embodiment of the invention.

FIG. 6B is a cross-sectional illustration of the liquid delivery system in FIG. 6A with an actuatable orifice that is in an open position, according to an embodiment of the invention.

FIG. 7 is a schematic of a computing device built in accordance with an embodiment of the invention.

### DETAILED DESCRIPTION OF THE INVENTION

Described herein are systems that include active venting systems and methods of forming such devices. In the following description, various aspects of the illustrative implementations will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative implementations. However, it will be apparent to one skilled in the art that the present invention may be practiced without the specific details. In other instances, well-known features are omitted or simplified in order not to obscure the illustrative implementations.

Various operations will be described as multiple discrete operations, in turn, in a manner that is most helpful in understanding the present invention, however, the order of description should not be construed to imply that these operations are necessarily order dependent. In particular, these operations need not be performed in the order of presentation.

As described above, providing vents in garments that can be opened and closed in order to regulate heat transfer is a desirable feature, particularly in athletic wear. However, currently available systems are bulky and prone to damage. Accordingly, embodiments of the present invention provide an active venting system that may be integrated into garments or other systems without substantially increasing the bulk of the garment or relying on damage prone fibers or stings. In order to obtain such an active venting system, piezoelectric material may be deposited on or adhered to the garment. Conductive traces formed on the piezoelectric material may then be used to apply a voltage differential across the piezoelectric material in order to allow for the piezoelectric material to strain, thereby opening a vent formed in the garment.

Particularly, embodiments of the invention are able to deposit the piezoelectric material onto the garment or other conformal substrate by using a pulsed laser annealing technique that does not result in the surrounding material being elevated to high temperatures. Previous piezoelectric materials required high temperature annealing processes. These processes are not suitable for fabrication of piezoelectric materials directly on substrates such as textiles or elastomeric substrates because the high temperatures would damage the substrates. As such, in order to utilize a piezoelectric actuator in conjunction with garments, the piezoelectric actuator would need to be fabricated as a discrete component on a high temperature substrate and then integrated into the textile, thereby increasing the bulk of the garment.

Additional embodiments of the invention utilize similar piezoelectric actuators integrated with other low-temperature materials to control the flow rate of fluids other than air. For example, some embodiments of the invention include a medicinal fluid sealed in a cavity. The cavity may have a liquid-tight opening that can be actuated with piezoelectric actuators similar to those used for garments described herein.

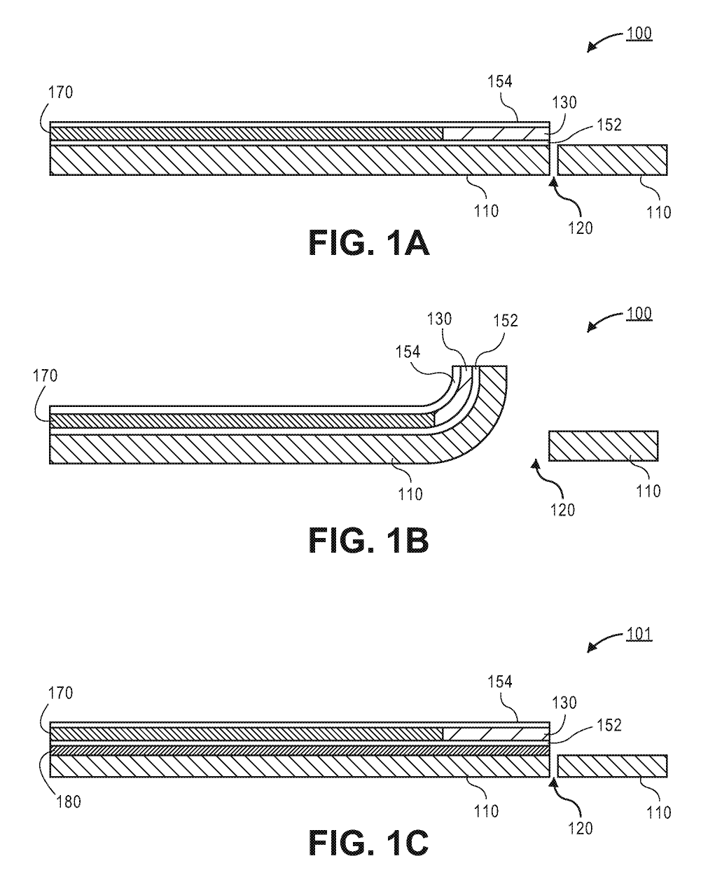

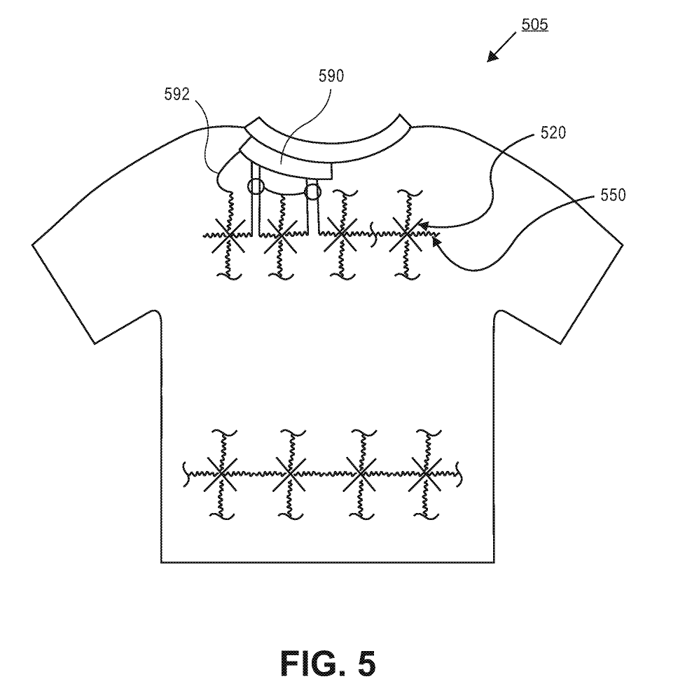

Referring now to FIG. 1A, a cross-sectional illustration of a portion of an active venting system 100 is shown, according to an embodiment of the invention. The active venting system 100 may be formed on any substrate 110 that is a conformal material that is able to flex, bend, stretch, and/or otherwise deform. In one particular embodiment, the substrate 110 may be a fabric. Fabrics may include organic fabrics (e.g., cotton, wool, etc.) and/or synthetic fabrics (e.g., nylon, rayon, polyester, etc.). In such embodiments, the fabric substrates 110 may be used to form garments (e.g., shirts, pants, undergarments, hats, etc.). An example of one such garment is described in greater detail below with respect to FIG. 5.

Additionally, while fabric substrates 110 used to form garments are described in greater detail, it is to be appreciated that the substrate 110 may be any suitable material that can be actuated in a substantially similar manner with piezoelectric actuation. Additionally, the products that employ such active venting may include any product that can benefit from the use of one or more controllable vents. For example, an active venting system 100 may be used in items such as blankets, window coverings, packaging (e.g., containers, bags, etc.), filtration systems, liquid dispensing systems, or the like.

Referring back to FIG. 1A, the substrate 110 includes a seam 120. In the illustrated embodiment, the seam 120 is shown as being a distinct gap through the substrate 110. However, it is to be appreciated that the seam 120 may not include any gap. For example, one portion of the substrate 110 may overlap the other portion. Alternatively, the surfaces of the substrate 110 along the seam 120 may be in contact with each other. The seam 120 may be any shape or pattern in the substrate 110 that allows for a vent to be opened by the actuator. Exemplary shapes and configurations for the seams 120 are described in greater detail below.

According to an embodiment, the actuation of the vent 120 may be driven by a piezoelectric actuator. The piezoelectric actuator may include a first electrode 152 and a second electrode 154. In one embodiment, the first electrode 152 may be formed on a different layer of the active venting system 100 than the second electrode 154. In such an embodiment, the first electrode 152 may contact a first surface of a piezoelectric material 130 and the second electrode 154 may contact a second surface of the piezoelectric material 130 that is opposite the first surface. According to an embodiment, the first electrode 152 may extend along a surface of the substrate 110 and the second electrode 152 may be separated from the first electrode 152 by an insulative layer 170. For example, the insulative layer 170 may be a conformal insulative material, such as a polymer.

The first and second electrodes 152, 154 may be electrically coupled to a voltage source (not shown). As such, a voltage differential across the first electrode 152 and the second electrode 154 may be generated. The voltage applied across the first electrode 152 and the second electrode 154 induces a strain in the piezoelectric layer 130 that causes displacement of the substrate 110. For example, in FIG. 1B, a voltage is applied and the substrate 110 is flexed upwards at the seam 120. Accordingly, the seam 120 opens to form a vent that allows for fluids and/or gasses to pass through the substrate 110. In an embodiment, the displacement of the substrate 110 is proportional to the voltage across the first electrode 152 and the second electrode 154. Since the displacement of the substrate 110 is proportional to the voltage applied to the first and second electrodes 152, 154, the seam 120 may be opened in an analog manner to different positions. As such, the amount of venting may be increased or decreased depending on the needs of the device.

According to an embodiment, the first electrode 152 and the second electrode 154 are formed with a conductive material. For example, the conductive material used for the first electrode 152 and the second electrode 154 may be any conductive material (e.g., copper, aluminum, alloys, etc.). In some embodiments, the conductive material may be printed onto the substrate 110 and/or the insulator 170.

In order to provide sufficient force to displace the substrate 110, embodiments of the invention include a high performance piezoelectric material for the piezoelectric layer 130. For example, the high performance piezoelectric layer 130 may be lead zirconate titanate (PZT), potassium sodium niobate (KNN), zinc oxide (ZnO), or combinations thereof. High performance piezoelectric materials such as these typically require a high temperature anneal (e.g., greater than 500° C.) in order to attain the proper crystal structure to provide the piezoelectric effect. As such, currently available piezoelectric actuators require a substrate that is capable of withstanding high temperatures (e.g., silicon). Low melting temperature substrates described herein, such as fabrics, typically cannot withstand such high temperatures. However, embodiments of the present invention allow for a piezoelectric layer 130 to be formed at much lower temperatures. For example, instead of a high temperature anneal, embodiments include depositing the piezoelectric layer 130 in an amorphous phase and then using a pulsed laser to crystalize the piezoelectric layer 130. For example, the piezoelectric layer 130 may be deposited with a sputtering process, an ink jetting process, or the like. According to an embodiment, the pulsed laser annealing process may use an excimer laser with an energy density between approximately 10-100 mJ/cm² and a pulsewidth between approximately 10-50 nanoseconds. Utilizing such an annealing process allows for the high performance piezoelectric layer 130 to be formed without damaging the substrate 110 on which the actuator is formed.

In order to enhance the adhesion between the deposited materials and the substrate 110, embodiments of the invention may also include an active venting system 101 that includes an intermediate layer 180, as illustrated in FIG. 1C. Some substrates 110 may not be particularly adapted to having conductive features or piezoelectric material deposited on them. In such an embodiment, an intermediate layer 180 may be used to improve the manufacturability of the actuator and/or improve the adhesion to the substrate 110. According to an embodiment, elastomeric materials or bonding tapes may be used as an intermediate layer 180. For example, elastomeric materials may be thermoplastic polyurethane (TPU), polydimethylsiloxane (PDMS), nitrile, Latex, or the like, and the bonding tape materials may include as polyimide, polyethylene terephthalate (PET), polyolefine (PO), or the like.

In addition to improved manufacturability, the use of an intermediate layer may provide additional benefits. For example, the actuation mechanism may be fabricated on a single material, which may later be integrated onto many different types of substrates 110 without needing to develop new processes for the different substrates 110. Instead a single process may be developed to fabricate the actuation mechanism on the intermediate layer 180, and the intermediate layer 180 may be laminated or otherwise attached to the different substrates 110. Additionally, a structure including only the intermediate layer 180, the first and second electrodes 152, 154, and the piezoelectric layer 130 may be sold independently to manufacturers that produce garments or other systems that would benefit from venting features. Accordingly, the manufacturers of these products do not need to have the technical or manufacturing capabilities to fabricate the piezoelectric actuator.

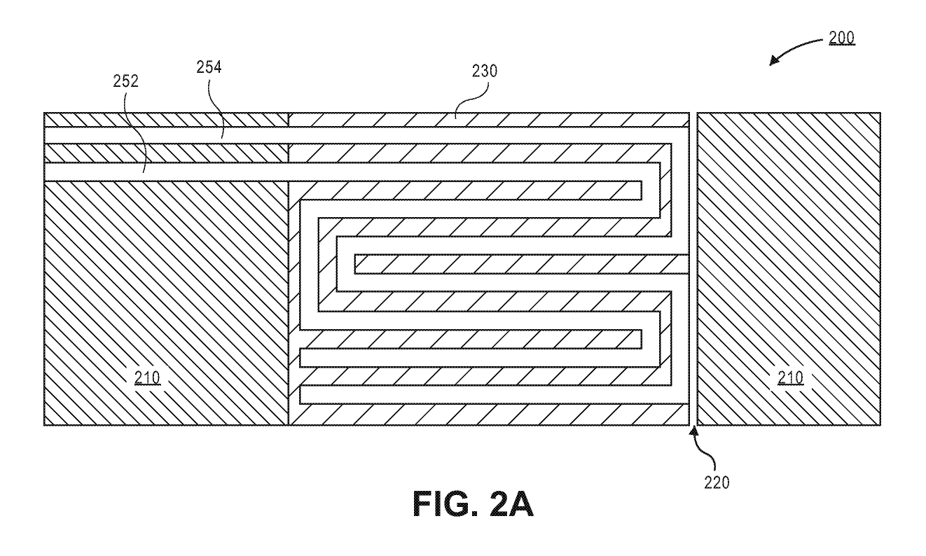

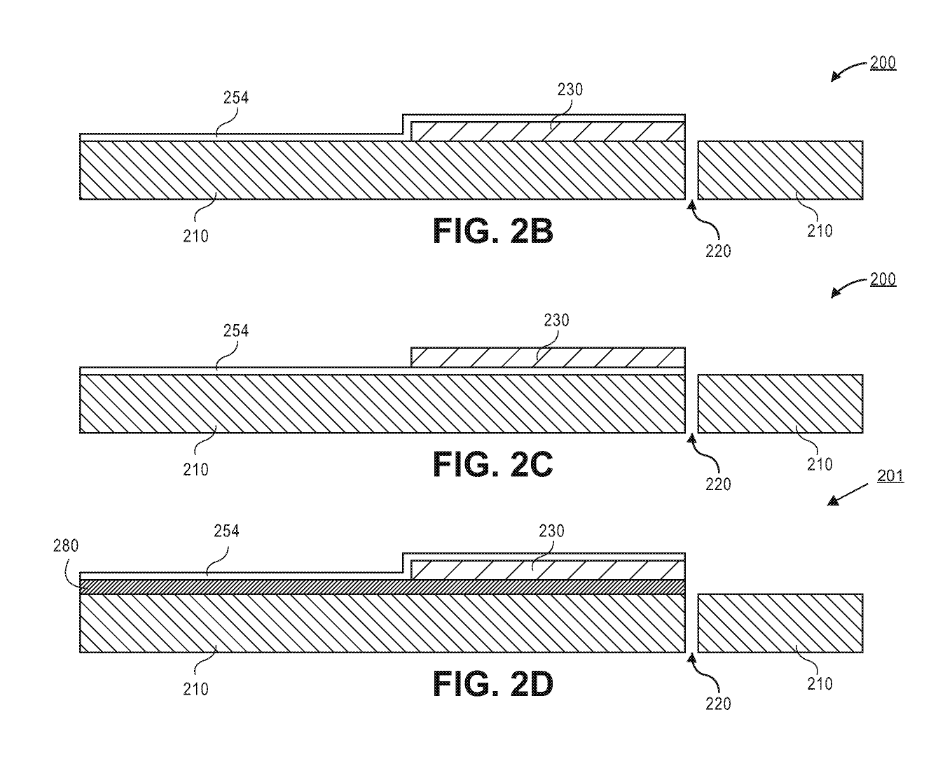

Referring now to FIG. 2A, a plan view of an active venting system 200 is illustrated according to an additional embodiment of the invention. In the illustrated embodiment, the first electrode 252 and the second electrode 254 are formed in a single layer. According to an embodiment, the first electrode 252 and the second electrode 254 may be formed in an interdigitated pattern. The interdigitated pattern allows for a voltage differential to be applied across the piezoelectric material 230 in order to initiate actuation of the substrate 210 at the seam 220. The interdigitated pattern illustrated in FIG. 2A is exemplary in nature, and it is to be appreciated that any desired interdigitated pattern may be used according to different embodiments of the invention.

Referring now to FIG. 2B, a cross-sectional illustration of the active venting system 200 is illustrated along a portion of the second electrode 254. As illustrated, the second electrode 254 extends over a top surface of the piezoelectric layer 230. Additionally, the first electrode (not visible in FIG. 2B) may also be formed over the top surface of the piezoelectric layer 230 in an interdigitated pattern with the second electrode 254. In an alternative embodiment, the electrodes may be formed below the piezoelectric material 230. Such an embodiment is illustrated in the cross-sectional view in FIG. 2C. As illustrated, the second electrode 254 is formed between the substrate 210 and the piezoelectric layer 230. The first electrode (not shown) may also be formed below the piezoelectric layer 230 in an interdigitated pattern with the second electrode 254.

According to yet another embodiment illustrated in FIG. 2D, an active venting system 201 may also include an intermediate layer 280 that may be formed between the substrate 210 and the piezoelectric material 230. Such an embodiment may be substantially similar to the embodiment illustrated in FIG. 1C, with the exception that the first and second electrodes are formed in a single layer. In the illustrated embodiment, the first electrode (not shown) and the second electrode 254 are formed over a top surface of the piezoelectric layer 230. However, embodiments may also include forming the first electrode and the second electrode below the piezoelectric layer 230, similar to the embodiment illustrated in FIG. 2C.

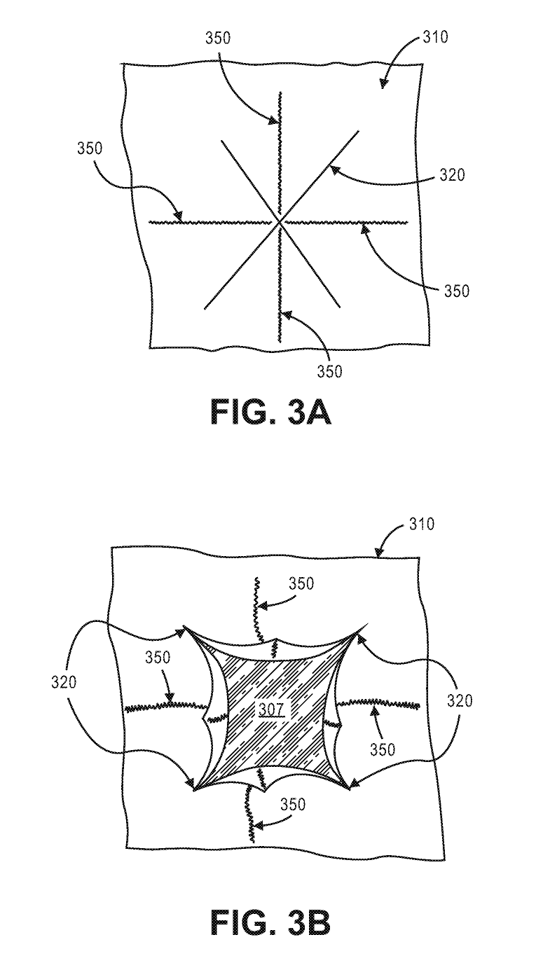

Referring now to FIGS. 3A and 3B, a more detailed illustration of how the vents are opened are shown, according to an embodiment of the invention. In FIG. 3A, a portion of the substrate 310 is shown. The substrate 310 may include a plurality of seams 320. According to an embodiment, the seams 320 may form an X-shape in the substrate 310. In such an embodiment, a piezoelectric actuator 350 may extend to the tip of each flap of the substrate 310 defined by the seams 320. For simplicity, the piezoelectric actuators 350 are illustrated as serpentine lines. However, it is to be appreciated that the piezoelectric actuators 350 may be substantially similar to the configurations described above with respect to FIGS. 1A-2D (e.g., a first electrode and a second electrode formed on opposing surfaces of a piezoelectric layer, or the first and second electrodes formed in a single layer in an interdigitated pattern over or under the piezoelectric layer). In FIG. 3A the vent is in a closed state (i.e., the piezoelectric actuators 350 are not activated).

Referring now to FIG. 3B, the actuators 350 are in an actuated state in order to open the vent along the seams 320. According to an embodiment, the actuators 350 cause each flap of the substrate 310 to bend out of plane of the substrate and expose a surface 307 below the substrate 320. In an embodiment, the surface 307 may be another textile. For example, the exposed surface may be a mesh material or other more permeable material. In some embodiments, the surface 307 may be integrated with the substrate 310 (e.g., a lining). Additional embodiments may include an underlying surface that is not integrated with the substrate 310. For example, the underlying surface 307 may be skin or an undergarment.

According to an embodiment, each of the actuators 350 may be connected in parallel and actuated in unison. Alternative embodiments may include actuators 350 that are independently controllable. In such embodiments, one or more of the actuators 350 may be activated in order to provide different sized openings. For example, if only a small vent is needed, then one of the four actuators 350 may be actuated. As such, the rate of cooling (e.g., air flow) through the substrate 310 may be controlled by activating one or more of the actuators 350.

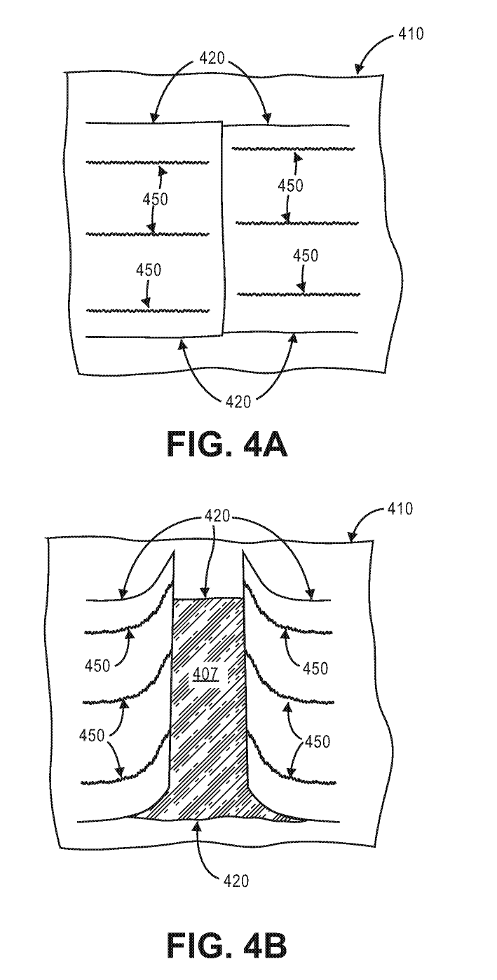

Referring now to FIGS. 4A and 4B, a detailed illustration of an additional configuration of vents in a substrate 410 are shown, according to another embodiment of the invention. In FIG. 4A, the seams 420 are configured so that flaps of the substrate 410 may be opened to form the vent. In the illustrated embodiment, the seams 420 are formed in an I-shape so that two flaps are formed in the substrate 410. However, it is to be appreciated that a single flap may be used. Depending on the size of the flap and the weight of the substrate 410, one or more actuators 450 may be used to provide sufficient force to open the flap. For example, in FIG. 4A, each flap includes three actuators 450.

Referring now to FIG. 4B, the actuators 450 are activated in order to open the vent and expose an underlying surface 407. The underlying surface 407 may be substantially similar to the underlying surface 307, and therefore, a detailed description will not be repeated here. In an embodiment, each of the one or more actuators 450 on each flap may be connected in parallel in order to operate in unison in order to form the vent. Additionally, when multiple flaps are formed proximate to each other, as illustrated in FIG. 4B, the one or more actuators 450 on each flap may be connected in parallel in order to operate in unison. Alternatively, the one or more actuators 450 on each flap may be operated independently. As such, the size of the vent through the substrate 410 may be controlled by only opening one flap at a time.

It is to be appreciated that the configuration of the seams illustrated in FIGS. 3A-4B are exemplary in nature. Additional embodiments of the invention may include any number of seams and/or flaps to define vents of any desired shape and/or size, depending on the needs of the system.

In FIGS. 3A-4B, a small portion of the substrate is shown. However, embodiments of the invention also include one or more substrates that are incorporated into a system level device. For example, one or more substrates may be fabricated into a garment or other larger article. FIG. 5 is an exemplary schematic illustration of one or more substrates that have been formed into a T-shirt 505. While a T-shirt 505 is shown, it is to be appreciated that any garment (e.g., pants, undergarments, hats, etc.), blankets, window coverings, packaging (e.g., containers, bags, etc.), filtration systems, liquid dispensing systems, or the like may utilize similar systems to provide active venting, in accordance with embodiments of the invention.

In FIG. 5 a T-shirt 505 with an active venting system is shown, according to an embodiment of the invention. In some embodiments, an electronics module 590 may provide electrical inputs to each of the actuators 550 in order to displace the substrate along a seam 520. While the seams 520 are illustrated as being substantially similar to those illustrated and described in FIGS. 3A-3B, it is to be appreciated that any seam configuration may be used. Additionally, the actuators 550 are illustrated schematically, and any actuation mechanism in accordance with embodiments of the invention may be used to open the vents in the T-shirt.

According to an embodiment, electrical connection between the electronics module 590 and the actuators 550 may be made with conductive traces 592. In order to simplify the Figure and not obscure embodiments of the invention, not all conductive traces 592 are shown. In an embodiment, the conductive traces 592 may be printed onto the material used to form the T-shirt 505. Additional embodiments of the invention may include conductive traces 592 that are conductive fibers that are sewn into the fabric of the T-shirt 505.

According to an embodiment, the electronics module 590 may include one or more processors that include logic for determining when to activate the actuators 550, which actuators 550 should be activated, and/or the amount of actuation for each actuator 550. In an embodiment, the electronics module 590 may be coupled to (or include) sensors. For example, the sensors may be used to determine body temperature, perspiration levels, heart rate, or the like of a person wearing the T-shirt 505. For example, if the body temperature rises above a threshold level, one or more of the actuators 550 may be activated in order to open vents for cooling the person wearing the T-shirt 505.

In addition to providing active venting for garments or other systems, embodiments of the invention may utilize active venting for control of other fluids. One such embodiment includes the controlled release of fluids for medicinal purposes. For example, a cavity that is filled with a medicinal fluid may have an orifice controlled by piezoelectric actuators. In such an embodiment, the medicinal fluid may be released at a controlled rate to provide more accurate control of medicine delivery.

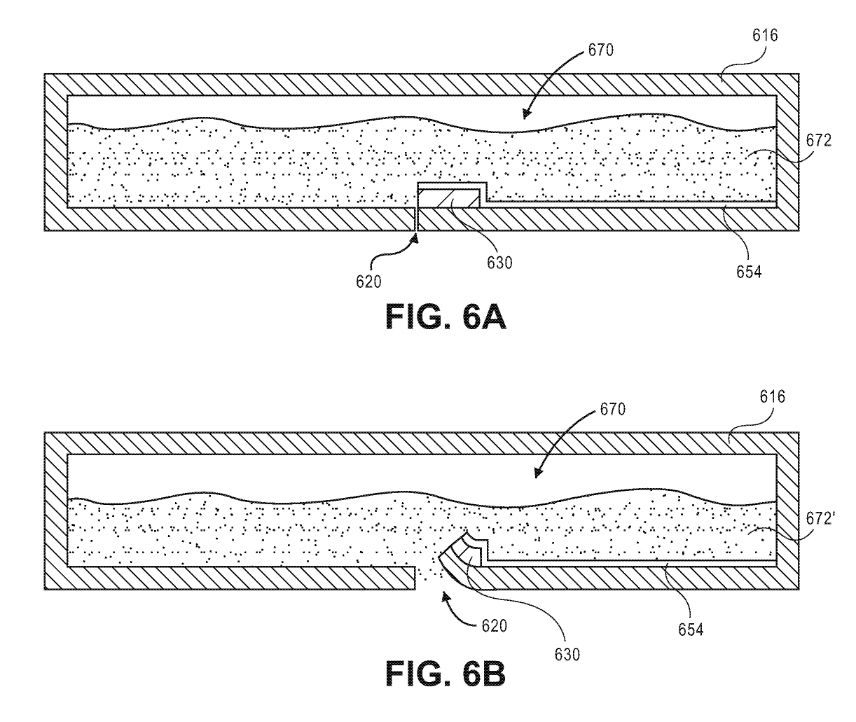

Referring now to FIGS. 6A and 6B, cross-sectional illustrations of a fluid delivery system 616 in a closed and an open state are shown, according to an embodiment of the invention. In an embodiment, the fluid delivery system 616 may form a cavity 670 that may be filled with a fluid 672. The fluid delivery system 616 may be any suitable material that may be substantially impermeable to the fluid 672 housed in the cavity. Alternative embodiments may include a fluid delivery system 616 that includes an impermeable coating (not shown) formed along the walls of the cavity 670. According to an embodiment, the cavity 670 may be sized so that a plurality of doses of the fluid 672 may be stored. As such, multiple doses may be released over a period of time (e.g., hours, days, weeks, etc.).

In order to allow for the release of the fluid 672, one or more seams 620 may be formed into the fluid delivery system 616. In an embodiment, the seams 620 may be substantially fluid-tight, so that the fluid 672 is not able to exit the cavity 670 when the piezoelectric layer 630 is not actuated. According to an embodiment, one or more seams 620 may be configured so that a plurality of openings into the cavity 670 may be formed upon actuation. In such embodiments, the rate of fluid 672 exiting the cavity may be increased or decreased to provide a desired dosage of the medicinal fluid 672. For example, if the medicinal fluid 672 is insulin, then different blood sugar levels may require different dosages that can be controlled by opening one or more vents into the cavity 670.

Referring now to FIG. 6B, the vent is opened along a seam 620 to allow for the fluid 672 to exit the cavity 670. For example, the fluid delivery system 616 may be placed in contact with the skin of a person (e.g., the fluid delivery system 616 may be a patch placed on a person's skin). In such an embodiment, the vent allows for fluid 672 to contact the skin. In order to allow for actuation, the piezoelectric layer 630 and the electrodes 654 may be formed in the interior of the cavity 670. The actuation of the piezoelectric layer 630 may cause a portion of the fluid delivery system 616 to curve back into the cavity 670 away from the surface on which the delivery system 616 is placed, thereby allowing for release of the fluid 672. As illustrated, the fluid 672′ remaining in the cavity may be less than the fluid 672 that was originally in the cavity 670 since some fluid has been released.

In the illustrated embodiment, the piezoelectric actuator is driven by a pair of interdigitated electrodes formed in a single layer, similar to those described above with respect to FIGS. 2A-2D. However, additional embodiments may include a piezoelectric actuator that is formed with electrodes in different layers similar to those described above with respect to FIGS. 1A-1C.

According to an embodiment, the actuation of the piezoelectric layer 630 may be controlled by an electronics module (not shown) that is substantially similar to the one described above with respect to FIG. 5. For example, the electronics module may utilize information obtained from one or more sensors to determine the required fluid flow needed. In the exemplary case of an insulin delivery system, the electronics module may receive information from a blood sugar sensor in order to determine the amount of insulin that should be delivered to a user. Additional embodiments may include an electronics module that actuates the openings at predetermined intervals. For example, a nicotine patch may provide a uniform dosage of nicotine at regular intervals.

While the fluid delivery system 616 in FIGS. 6A and 6B is described in view of delivering medicinal fluids, it is to be appreciated that embodiments are not limited to such configurations. For example, a fluid delivery system 616 that includes piezoelectric actuators may be used to control the release rate of any fluid. Such embodiments may be utilized for industrial applications where a controlled release of a fluid is needed.

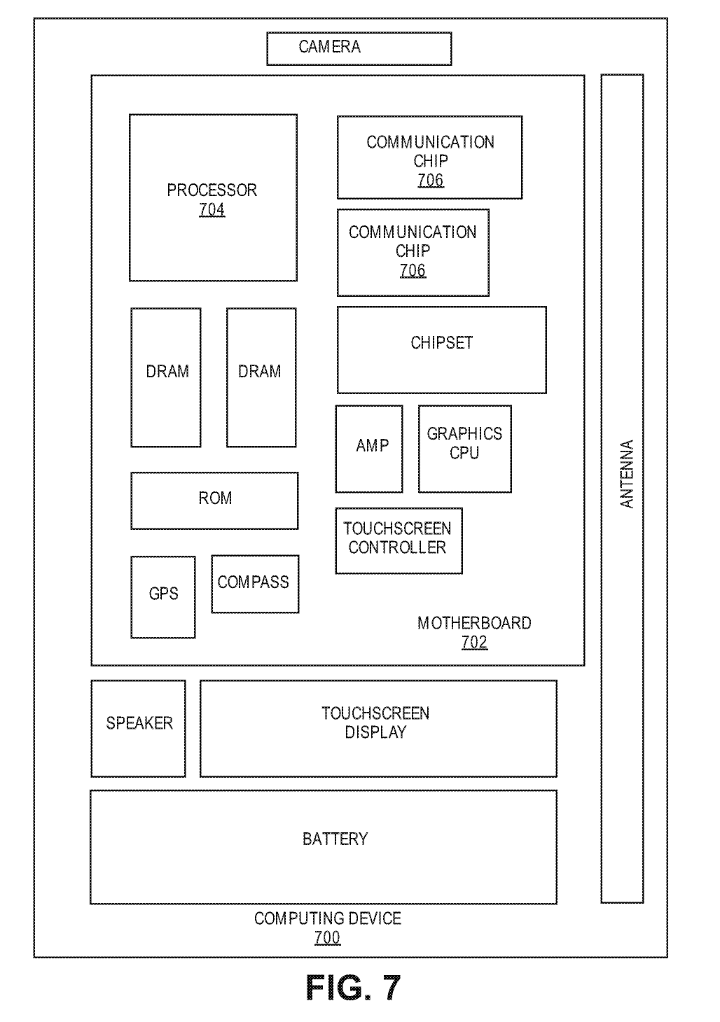

FIG. 7 illustrates a computing device 700 in accordance with one implementation of the invention. The computing device 700 houses a board 702. The board 702 may include a number of components, including but not limited to a processor 704 and at least one communication chip 706. The processor 704 is physically and electrically coupled to the board 702. In some implementations the at least one communication chip 706 is also physically and electrically coupled to the board 702. In further implementations, the communication chip 706 is part of the processor 704.

Depending on its applications, computing device 700 may include other components that may or may not be physically and electrically coupled to the board 702. These other components include, but are not limited to, volatile memory (e.g., DRAM), non-volatile memory (e.g., ROM), flash memory, a graphics processor, a digital signal processor, a crypto processor, a chipset, an antenna, a display, a touchscreen display, a touchscreen controller, a battery, an audio codec, a video codec, a power amplifier, a global positioning system (GPS) device, a compass, an accelerometer, a gyroscope, a speaker, a camera, and a mass storage device (such as hard disk drive, compact disk (CD), digital versatile disk (DVD), and so forth).

The communication chip 706 enables wireless communications for the transfer of data to and from the computing device 700. The term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. The communication chip 706 may implement any of a number of wireless standards or protocols, including but not limited to Wi-Fi (IEEE 802.11 family), WiMAX (IEEE 802.16 family), IEEE 802.20, long term evolution (LTE), Ev-DO, HSPA+, HSDPA+, HSUPA+, EDGE, GSM, GPRS, CDMA, TDMA, DECT, Bluetooth, derivatives thereof, as well as any other wireless protocols that are designated as 3G, 4G, 5G, and beyond. The computing device 700 may include a plurality of communication chips 706. For instance, a first communication chip 706 may be dedicated to shorter range wireless communications such as Wi-Fi and Bluetooth and a second communication chip 706 may be dedicated to longer range wireless communications such as GPS, EDGE, GPRS, CDMA, WiMAX, LTE, Ev-DO, and others.

The processor 704 of the computing device 700 includes an integrated circuit die packaged within the processor 704. In some implementations of the invention, the integrated circuit die of the processor may be packaged on a substrate or garment that includes one or more seams and one or more piezoelectric actuators for opening vents, in accordance with implementations of the invention. The term “processor” may refer to any device or portion of a device that processes electronic data from registers and/or memory to transform that electronic data into other electronic data that may be stored in registers and/or memory.

The communication chip 706 also includes an integrated circuit die packaged within the communication chip 706. In accordance with another implementation of the invention, the integrated circuit die of the communication chip may be packaged on a substrate or garment that includes one or more seams and one or more piezoelectric actuators for opening vents, in accordance with implementations of the invention.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize.

These modifications may be made to the invention in light of the above detailed description. The terms used in the following claims should not be construed to limit the invention to the specific implementations disclosed in the specification and the claims. Rather, the scope of the invention is to be determined entirely by the following claims, which are to be construed in accordance with established doctrines of claim interpretation.

Embodiments of the invention include an active venting system, comprising: a substrate having one or more seams formed through the substrate; a piezoelectric layer formed proximate to one or more of the seams; and a first electrode and a second electrode contacting the piezoelectric layer.

Additional embodiments of the invention include an active venting system, wherein the first electrode contacts a first surface of the piezoelectric layer and the second electrode contacts a second surface of the piezoelectric layer that is opposite to the first surface of the piezoelectric layer.

Additional embodiments of the invention include an active venting system, further comprising an insulative layer formed between the first electrode and the second electrode.

Additional embodiments of the invention include an active venting system, wherein the first electrode and the second electrode are formed along a single surface of the piezoelectric layer.

Additional embodiments of the invention include an active venting system, wherein the first electrode and the second electrode are interdigitated.

Additional embodiments of the invention include an active venting system, wherein the first electrode and the second electrode are formed above the piezoelectric layer.

Additional embodiments of the invention include an active venting system, wherein the first electrode and the second electrode are formed between the substrate and the piezoelectric layer.

Additional embodiments of the invention include an active venting system, further comprising an intermediate layer between the substrate and the piezoelectric layer.

Additional embodiments of the invention include an active venting system, wherein the intermediate layer is an elastomer or a bonding tape.

Additional embodiments of the invention include an active venting system, wherein the one or more seams define one or more flaps in the substrate.

Additional embodiments of the invention include an active venting system, wherein the one or more seams form an X-shape pattern or an I-shape pattern.

Additional embodiments of the invention include an active venting system, wherein each flap includes a piezoelectric layer and first and second electrodes.

Additional embodiments of the invention include an active venting system, wherein the first and second electrodes on each flap are independently controllable.

Additional embodiments of the invention include an active venting system, wherein the first and second electrodes on each flap are controlled in parallel.

Additional embodiments of the invention include an active venting system, wherein the substrate is a textile.

Additional embodiments of the invention include an active venting system, wherein the piezoelectric material is zirconate titanate (PZT), potassium sodium niobate (KNN), or zinc oxide (ZnO).

Embodiments of the invention include an active venting garment comprising: a textile material; an electronics module integrated with the textile material; and one or more vents formed into the textile material, wherein each vent comprises: one or more seams formed through the textile material; a piezoelectric layer formed proximate to one or more of the seams; and a first electrode and a second electrode contacting the piezoelectric layer and electrically coupled to the electronics module.

Additional embodiments of the invention include an active venting garment, wherein the electronics module is electrically coupled to the first and second electrodes by conductive fibers integrated into the textile material.

Additional embodiments of the invention include an active venting garment, wherein each of the first and second electrodes are independently controllable.

Additional embodiments of the invention include an active venting garment, further comprising an intermediate layer between the textile and the piezoelectric layer.

Additional embodiments of the invention include an active venting garment, wherein the intermediate layer is an elastomer or a bonding tape.

Embodiments of the invention include a fluid delivery system, comprising: a cavity having one or more fluid-tight seams; a piezoelectric material formed along a surface of the cavity proximate to the one or more fluid-tight seams; and a first electrode and a second electrode contacting the piezoelectric layer.

Additional embodiments of the invention include a fluid delivery system, wherein the one or more fluid-tight seams allows for a portion of the fluid delivery system to displace into the cavity upon actuation of the piezoelectric material by the first and second electrode.

Additional embodiments of the invention include a fluid delivery system, wherein the cavity contains a medicinal fluid.

Additional embodiments of the invention include a fluid delivery system, wherein the cavity is lined with a layer substantially impermeable to the medicinal fluid.

What is claimed is:

1. An active venting system, comprising:
a substrate having one or more seams formed through the substrate;
a piezoelectric layer formed proximate to one or more of the seams; and
a first electrode and a second electrode contacting the piezoelectric layer.

2. The active venting system of claim 1, wherein the first electrode contacts a first surface of the piezoelectric layer and the second electrode contacts a second surface of the piezoelectric layer that is opposite to the first surface of the piezoelectric layer.

3. The active venting system of claim 2, further comprising an insulative layer formed between the first electrode and the second electrode.

4. The active venting system of claim 1, wherein the first electrode and the second electrode are formed along a single surface of the piezoelectric layer.

5. The active venting system of claim 4, wherein the first electrode and the second electrode are interdigitated.

6. The active venting system of claim 4, wherein the first electrode and the second electrode are formed above the piezoelectric layer.

7. The active venting system of claim 4, wherein the first electrode and the second electrode are formed between the substrate and the piezoelectric layer.

8. The active venting system of claim 1, further comprising an intermediate layer between the substrate and the piezoelectric layer.

9. The active venting system of claim 8, wherein the intermediate layer is an elastomer or a bonding tape.

10. The active venting system of claim 1, wherein the one or more seams define one or more flaps in the substrate.

11. The active venting system of claim 10, wherein the one or more seams form an X-shape pattern or an I-shape pattern.

12. The active venting system of claim 10, wherein each flap includes a piezoelectric layer and first and second electrodes.

13. The active venting system of claim 12, wherein the first and second electrodes on each flap are independently controllable.

14. The active venting system of claim 12, wherein the first and second electrodes on each flap are controlled in parallel.

15. The active venting system of claim 1, wherein the substrate is a textile.

16. The active venting system of claim 1, wherein the piezoelectric material is zirconate titanate (PZT), potassium sodium niobate (KNN), or zinc oxide (ZnO).

17. An active venting garment comprising:
a textile material;
an electronics module integrated with the textile material; and
one or more vents formed into the textile material, wherein each vent comprises:
one or more seams formed through the textile material;
a piezoelectric layer formed proximate to one or more of the seams; and
a first electrode and a second electrode contacting the piezoelectric layer and electrically coupled to the electronics module.

18. The active venting garment of claim 17, wherein the electronics module is electrically coupled to the first and second electrodes by conductive fibers integrated into the textile material.

19. The active venting garment of claim 17, wherein each of the first and second electrodes are independently controllable.

20. The active venting garment of claim 17, further comprising an intermediate layer between the textile and the piezoelectric layer.

21. The active venting system of claim 20, wherein the intermediate layer is an elastomer or a bonding tape.

22. A fluid delivery system, comprising:
a cavity having one or more fluid-tight seams;
a piezoelectric material formed along a surface of the cavity proximate to the one or more fluid-tight seams; and
a first electrode and a second electrode contacting the piezoelectric layer.

23. The fluid delivery system of claim 23, wherein the one or more fluid-tight seams allows for a portion of the fluid delivery system to displace into the cavity upon actuation of the piezoelectric material by the first and second electrode.

24. The fluid delivery system of claim 23, wherein the cavity contains a medicinal fluid.

25. The fluid delivery system of claim 24, wherein the cavity is lined with a layer substantially impermeable to the medicinal fluid.
